(19) United States
(12) Patent Application Publication
(10) Pub. No.: US 2022/0062126 A1
(43) Pub. Date: Mar. 3, 2022

(54) UV SCREENING AND ANTIOXIDANT COMPOSITION AND USE THEREOF

(71) Applicant: BEIERSDORF AG, Hamburg (DE)

(72) Inventors: Eduardo C. RUVOLO, Flanders, NJ (US); Thomas A. MEYER, Germantown, TN (US)

(21) Appl. No.: 17/420,227

(22) PCT Filed: Jan. 2, 2020
(86) PCT No.: PCT/US2020/012051

§ 371 Date: Jul. 1, 2021

Related U.S. Application Data
(60) Provisional application No. 62/788,418, filed on Jan. 4, 2019.

(51) Int. Cl. A61K 8/27; A61K 8/37; A61K 8/67; A61Q 17/04; A61Q 19/08

(52) U.S. Cl. CPC A61K 8/27; A61K 8/37; A61K 8/676; A61K 8/678; A61K 2800/592; A61Q 19/08; A61K 2800/412; A61K 2800/522; A61Q 17/04

(57) ABSTRACT

The present disclosure relates generally to antioxidant compositions and their uses in personal care products. More specifically, the disclosure provides diethylhexyl syringylidene malonate-containing compositions for use in sunscreen or non-sun-screen formulations. Also provided are diethylhexyl syringylidene malonate-containing compositions and methods for treating and/or preventing photodamage caused by long UVA and/or visible light.

### TECHNICAL FIELD

The present disclosure relates generally to antioxidant compositions and their uses in personal care products. More specifically, the disclosure provides compositions containing a particular combination of antioxidants comprising diethythexyl syringylidene malonate, which form a “super antioxidant combination”, suitable for use in either sunscreen or non-sunscreen formulations. Also provided are such antioxidant compositions and methods for treating and/or preventing photodamage from long Ultraviolet A (from 370 nm to 400 nm) and visible light.

### DISCUSSION OF BACKGROUND INFORMATION

It has been long recognized that sunlight exposure increases skin damage. Although the contribution of UV light to skin damage has been established, few studies have examined the effects of longer wavelength radiation, such as visual light (400 nm-700 nm). Irradiation of the skin with visible light induces production of reactive oxygen species (ROS), proinflammatory cytokines, and matrix metalloproteinase (MMP)-1 expression. Commercially available sunscreens are generally found to have minimal effects on reducing visible light-induced ROS, suggesting that UVA/UVB sunscreens do not protect the skin from long UVA (370 to 400 nm) and visible light.

Therefore, there exists a need in the art for compositions that protect skin from long UVA and/or visible light in sunlight and/or in artificial lighting.

### SUMMARY OF THE INVENTION

Provided herein are antioxidant compositions that meet the need above, and such compositions may be used in personal care products, like sunscreens. In some aspects, the present disclosure is directed to antioxidant compositions suitable for use alone or in combination with any agents in protecting skin, as well as treating and/or preventing photodamage by long Ultraviolet A (UVA) and/or visible light. Such antioxidant compositions can reduce skin darkening, reduce or treat melanogenesis, reduce free radical production, reduce photoaging, and/or reducing reactive oxygen species (ROS). It has been contemplated that compositions disclosed herein may be used to provide prophylactic as well as therapeutic treatments for skin conditions.

In some aspects, provided are antioxidant compositions comprising a super antioxidant combination, In some embodiments, provided is an antioxidant composition that comprises diethylhexyl syringylidene malonate and other antioxidants. In one variation, the other antioxidants can he selected from vitamin E vitamin C, and a combination thereof. In certain embodiments, the super antioxidant combination consists of diethythexyl syringylidene malonate, vitamin E and vitamin C. In other embodiments, the super antioxidant combination consists essentially of diethylhexyl syringylidene malonate, vitamin E and vitamin C.

In some aspects, provided are antioxidant compositions and uses of such antioxidant compositions in personal care products. In some embodiments, provided personal care composition that comprises the antioxidant composition and at least one additional ingredient. In some embodiments, such antioxidant compositions are used. in sunscreen or non-sunscreen formulations. In some embodiments, at least one additional ingredient comprises a sunscreen active and such a personal care composition is a sunscreen product. In one embodiment, a personal care composition is not marketed as a sunscreen product (although it may contain sunscreen actives).

In other aspects, provided are methods for preventing and/or treating photodamage induced by long UVA andlor visible light comprising topically applying the compositions as described herein.

In yet other aspects, the present disclosure provides a method for confirming antioxidant activity of a composition formulated for topical application to skin, wherein the method comprises clinically testing the composition for ability to reduce photodamage induced by long UVA and/or visible light. Also provided is a method for screening compositions to be topically applied to skin for antioxidant behavior, wherein the screening method comprises determining the composition's ability to reduce photodamage induced by long UVA and/or visible light.

In another aspect, provided is also an article of manufacture, such as a container, comprising any of the compositions described herein, and a label containing instructions for use of the composition.

### BRIEF DESCRIPTION OF THE DRAWINGS

The present application can be best understood by reference to the following description taken in conjunction with the accompanying drawings included in the specification.

FIG. 1 shows the exposure sites on a human subject. Marked areas were exposed to light irradiation t a dose of 320 J/cm² on all sites.

FIG. 2 shows pigmentation on African Americans subjects (from left to right: immediate, 24 hours and 7 days).

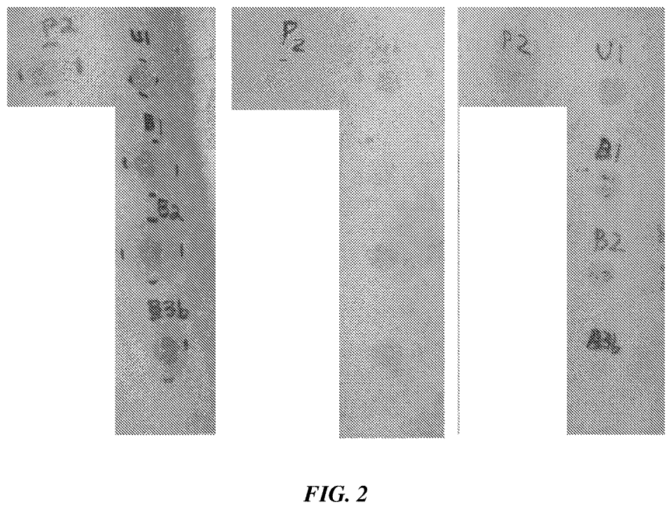

FIG. 3 illustrates the difference in the mean melanin content in skin (exposed-non exposed) for the 10 dark skin Phototypes (IV-VI) for each time point.

FIG. 4 illustrates the difference in the mean erythema content in skin (exposed-non exposed) for the 10 light skin Phototypes for each time point.

### DETAILED DESCRIPTION OF EMBODIMENTS OF THE INVENTION

The following description sets forth exemplary methods, parameters and the like. It should be recognized, however, that such description is not intended as a limitation on the scope of the present disclosure but is instead provided as a description of exemplary embodiments,

As used herein, the word “a”, “an” or “plurality” before a noun represents one or more of the particular noun. For the terms “for example” and “such as,” and grammatical equivalences thereof, the phrase “and without limitation” is understood to follow unless explicitly stated otherwise. As used herein, the term “about” is meant to account for variations due to experimental error. measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used.

All percentages and ratios used herein are by weight of the total composition and all measurements made are at 25′ C. unless otherwise designated.

The antioxidant compositions as described herein can comprise any form readily known by those of ordinary skill in the art of preparing personal care compositions. Examples of such include, but are not limited to, nonionic vesicle dispersions, emulsions, creams, milks, gets, cream gels, ointments, suspensions, dispersions, powders, solids, sticks, foams or sprays. In some embodiments, the composition can comprise an anhydrous or aqueous solid or paste, emulsion, suspension, or dispersion. Exemplary forms of the compositions include an oil-in-water emulsion, a water-in-oil emulsion, an alcohol solution, or an aerosol formulation.

### Antioxidant Components

In some aspects, provided are antioxidant compositions including a super antioxidant combination. In some embodiments, the super antioxidant combination comprises diethylhexyl syringylidene malonate and other antioxidants. In one variation, the super antioxidant combination comprises diethylhexyl syringylidene malonate and vitamin E. In another variation, the super antioxidant combination comprises diethylhexyl syringylidene malonate and vitamin C. In yet another variation, the super antioxidant combination comprises diethylhexyl syringylidene malonate, vitamin E and vitamin C.

Examples of antioxidants useful in the compositions include, but are not limited to, Vitamin E, vitamin C diisopropyi vanillidene malonate (also referred to as DIPVM) and related compounds (described in U.S. Pat. Nos. 6,602,515; 6,831,191; 6,936,735; 7,150,876; and 7,166,273), Tetrahydrocurcumenoids, Soybean zymbiozome fermentum, Red clover extract, Vitis vinifera (grape) seed extract/Brand B, Green tea extract, Pikea robusta extract, Tocopherol (and) Vitis vinifera (grape) seed extract, Vitis vinifera (grape) seed extract/Brand A, Phylanthus emblica fruit extract and combinations thereof.

Diethylhexyl Syringylidene Malonate

The compositions described herein comprise a safe and effective amount of diethylhexyl syringylidene malonate. In one variation, the diethylhexyl syrinylidene malonate is or comprises Oxynex ST® (Merck KgaA) which exhibits quenching and anti-oxidant properties.

In some embodiments, diethylhexyl syringylidene malonate is present in an amount of from about 1% to about 10% by weight based on a total weight of the composition. In some embodiments, diethylhexyl syringylidene malonate is present in an amount of from about 1% to about 5% by weight of the composition. In some embodiments, diethylhexyl syringylidene malonate is present in an amount of from about 1% to about 2% by weight of the composition. In some embodiments, diethylhexyl syringylidene malonate is present in an amount of about 1%, about 1.5%, about 2%, about 3%, about 5 or about 10% by weight of the composition. In some embodiments, diethylhexyl syringylidene malonate is present in an amount of at least 1% by weight of the composition. In some embodiments, diethylhexyl syringyridene malonate is present in an amount of greater than 0.5% by weight of the composition.

Vitamins

In addition to diethylhexyl syringylidene malonate, other antioxidants can be used in the antioxidant composition. In some embodiments, the antioxidant compositions comprise diethylhexyl syringylidene malonate and one or more vitamins. In some embodiments, the vitamins are selected from vitamin E, vitamin C, and combination thereof. In certain embodiments, the antioxidant composition is a super antioxidant composition consisting essentially of diethylhexyl syringylidene malonate, vitamin E and vitamin C.

In some variations, the antioxidant composition described herein contains vitamin E. In some embodiments, vitamin E is present in an amount of from about 5% to about 0.001% by weight based on a total weight of the composition. In some embodiments, vitamin E is present in an amount of from about 5% to about 0.001% by weight. In some embodiments, vitamin E is present in an amount of from about 2% to about 0.01% by weight. In some embodiments, vitamin E is present at about 2%, about 1%, about 0.5%, about 0.25%, about 0.1%, about 0.05.%, about 0.02%, or about 0.01% by weight. In some embodiments, vitamin E is present at about 0.25% by weight.

In some variations, the antioxidant composition described herein contains vitamin C. In some embodiments, vitamin C is present in an amount of from about 5% to about 0.001% by weight based on a total weight of the composition. In some embodiments, vitamin C is present in an amount of from about 2% to about 0.01% by weight. In some embodiments, vitamin C is present at about 2%, about 1%, about 0.5%, about 0.25%, about 0.1%, about 0.05%, about 0.02%, or about 0.01% by weight. In some embodiments, vitamin C is present at about 0.01% by weight.

In some embodiments, the antioxidant composition comprises at least 1% diethylhexyl syringylidene malonate, as well as vitamin E and vitamin C. In some embodiments, the antioxidant composition comprises 1% diethylhexyl syringylidene malonate, as well as vitamin E and vitamin C. In some embodiments, the antioxidant composition comprises 2% diethylhexyl syringylidene malonate, as well as vitamin E and. vitamin C. In some embodiments, the antioxidant composition comprises 1% or 2% diethylhexyl syringylidene malonate, as well as 0.25% vitamin E and 0.01% vitamin C.

Additional Ingredients

The present disclosure also provides an antioxidant composition used as a personal care composition for topical application to the skin and/or hair, comprising an antioxidant described herein and/or combinations thereof and amount of antioxidants determined by the methods described herein. Non-limiting examples of such personal care compositions may include such products as moisturizers, cleansers, conditioners, shampoo, body wash, styling gell/lotion, eye cream and eye liner, facial cream, blush, mascara, foundation, nail polish, polish remover, eye shadow, lipstick, lip gloss, lip liners, lip balms, makeup remover, nail treatment, foot care compositions, acne treatment, redness/rosacea treatment, varicose/spider vein treatment, anti-aging compositions, sunless tanning compositions, after-sun compositions, concealers, hair color and bleaching compositions, skin fading/lighteners, body firming lotion, shaving cream, after shave, relaxer, antiperspirants and deodorants, exfoliants, scrubs, liquid hand soap, bubble bath, pain and wound treatment compositions, insect repellant, anti-itch and rash cream, styling mousse and foams, perfume, lubricants, body oil, body spray, baby lotion, diaper cream, baby soap, baby shampoo, baby oil, baby wipes, hair-loss treatment, hair spray, depilatory, hair growth inhibitors, hair removal waxes, personal cleansing, cologne, oil controller, and hand sanitizer.

In accordance to some embodiments of the present disclosure, the composition described herein comprises an antioxidant described herein and/or combinations thereof and at least one additional ingredient.

Such compositions typically comprise a safe and effective amount of at least one additional skin and/or hair care active. A representative, non-limiting list of such actives includes sunscreen actives, sugar amines, retinoids, hydroquinone, peptides, famesol, phytosterol, dialkanoyl hydroxyproline, hexamidine, salicylic acid, N-acyl amino acid compounds, glycyrrhizic acid, glycyrrhetinic acid, camosine, Butylated Hydroxytoluene (BHT) and Butylated Hydroxyanisole (BHA), menthyl anthranilate, cetyl pyridinium chloride, tetrahydrocurmin, vanillin or its derivatives, ergothioneine, melanostatin, sterol esters, idebenone, dehydroacetic acid, yeast extract (e.g,, Pitera®), beta Oilcans, alpha glucans, fatty acids (especially poly-unsaturated fatty acids), zinc pyrithione (ZPT), anti-fungal agents, thiol compounds (e.g., N-acetyl cysteine, glutathione, thioglycolate), beta-carotene, ubiquinone, amino acids, their salts, their derivatives, their precursors, and/or combinations thereof.

The compositions of the present disclosure may contain a variety of other ingredients that are conventionally used in given product types provided that they do not unacceptably alter the benefits of the invention.

Such additional ingredient(s), when incorporated into the composition, should be suitable for use in contact with human skin without undue toxicity, incompatibility, instability, allergic response, and the like within the scope of sound judgment, The CITA Cosmetic Ingredient Handbook, Seventh Edition (1992) and the English Edition (2000), which is incorporated by reference herein in its entirety, describes a wide variety of nonlimiting cosmetic and pharmaceutical ingredients conunonly used in the skin care industry, which are suitable for use in the compositions of the present invention. Examples of these ingredient classes include: abrasives, absorbents, aesthetic components such as fragrances, pigments, colorings/colorants, essential oils, anti-caking agents, antifoaming agents, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g, copolymer of eicosene and vinyl pyrrolidone), opacifying agents, pH adjusters, propellants, reducing agents, sequestrants, and thickeners.

Additional ingredients useful in the present invention include those described in U.S. Publication No. 200410175347A1, including desquamation actives, such as salicylic acid and zwitterionic surfactants; soothing and/or healing agents; skin treating agents; skin sensates, astringents, etc. (e.g., clove oil, menthol, camphor, eucalyptus oil, eugenol, menthyl lactate, witch hazel distillate); anti-acne actives, such as resorcinol, sulfur, erythromycin, zinc, dehydroacetic acid; anti-wrinkle actives/anti-atrophy actives; anti-oxidants/radical scavengers, such as tocopherol; chelators, such as furildioxime and derivatives thereof; flavonoids; anti-inflammatory agents; anti-cellulite agents; tanning actives such as dihydroxyacetone; skin lightening agents; antimicrobial and antifungal actives; sunscreen actives; conditioning agents such as glycerol, urea, petrolatum, sucrose polyester, and combinations thereof; thickening agents such as carboxylic: acid polymers, crosslinked polyacrylate polymers, polyacrylamide polymers, polysaccharides, gums; water-soluble vitamins; and particulate materials. Compositions of the present invention may contain a safe and effective amount of one or more of the following other actives or ingredients: fatty acids (especially poly-unsaturated fatty acids), glucosamine, zinc pyrithione (ZPT), thiol compounds (e.g., N-acetyl cysteine, glutathione, thioglycolate), other vitamins (e.g., B1, B2, B5,B6, B12, D, F, K, P), beta-carotene, ubiquinone, if,iebenone, amino acids, minerals (e.g., Zn, Mn, Mg, Cu, Fe, and Se), hydroxy acids (e.g., alpha-hydroxy acids, alpha-keto acids, and beta-hydroxy acids), kojic aid, arbutin, mulberry extract, exfoliation agents, anti-dandruff agents, and the like.

In some embodiments, at least one additional ingredient is an anti--aging ingredient such as retinol, retinoic acid, and alpha hydroxy acids (AHA) (e.g. glycolic and lactic acids). In some embodiments, at least one additional ingredient is a depigmenting agent such as hexyl resorcinol, azelaic acid and hydroquinone. In some embodiments, at least one additional ingredient is a sunscreen active. A non-sunscreen active ingredient can be used alone or together with sunscreen actives in compositions provided herein.

Sunscreen Actives

The compositions provided herein may be suitable for use as either sunscreen or non-sunscreen formulations. In one variation, the compositions are suitable for use as sunscreen formulations, and as such, the compositions further comprise sunscreen actives)

In some embodiments, compositions as described herein contain mineral sunscreen actives. In some embodiments, compositions contain both mineral sunscreen actives and organic actives, Suitable organic sunscreen actives may include, without limitation, para-aminobenzoic acid, avobenzone, cinoxate, dioxybenzone, homosalate, menthyl anthranilate, octocrylene, octyl rnethoxycinnamate, octyl salicylate, oxybenzone, 2-ethylhexyl 4-(dimethylamino)benzoate (e.g., Padimate O), phenylbenzimidazole sulfonic acid, octisalate, sulisobenzone, and trolamine salicylate. In one variation, compositions described herein contain titanium dioxide (a mineral active) and avobenzone (an organic active). In one variation, compositions described herein contain zinc oxide (a mineral active) and avobenzone (an organic active). In another variation, compositions described herein contain zinc oxide (a mineral active) and titanium dioxide (a mineral active) and avobenzone (an organic active),

In certain embodiments, the compositions use only mineral sunscreen actives such as zinc oxide and titanium dioxide, and exclude organic actives.

In some embodiments, the mineral sunscreen actives used are zinc oxide. In certain embodiments, the compositions provided are all mineral sunscreen compositions made up of zinc oxide, and have unexpectedly been found to achieve a Sun Protection Factor (SPF) of at least 50.

The composition, in some embodiments, is formulated as a hybrid water-in-silicone/oil inverted emulsion. In addition to antioxidant components as described herein, the composition also contains no more than 25 wt % zinc oxide (based on a total weight of the composition), wherein the zinc oxide is surface treated. Such composition can further include film formers, SPF boosters, and other ingredients. The composition, the sunscreen actives and other agents used therein, as well as the method of manufacturing such compositions are described in further detail below.

In certain embodiments, the zinc oxide is surface-treated. Generally speaking, surface treatment enhances the quality of zinc oxide dispersions and increases the proportion of zinc oxide that can be successfully loaded into emulsions. If desired, the surface treatment can be selected according to the nature of the intended end product, some options for which are known to those skilled in the art. Exemplary surface treatment agents include, but are not limited to, metliicone, hydrogen dimethicone, dimethicone, triethoxycaprylylsilane, jojoba. esters, poiymethylsilsesquioxa.ne, magnesium myristate (and other metal soaps), stearic acid, stearoyl glutamic acid (and other amino acid treatments), lecithin, silica, sodium polyacrylate, PEG-10 dimethicone, sodium lauroyl aspartate, stearyl triethoxysilane, disodium carboxyethyl siticonate, trimethylsiloxysthcate, dimethicone PEG-3 laurate, stearyl triethoxysilane, disodium carboxyethyl siliconate, trimethylsiloxysilicate, polycaprylylsilsesquioxane, polydiethylsiloxane, triethoxysilylpropyl acetyl hydroxyprolinate, perfluorooctylethyltriethoxysilane, methoxy PEG-10 propyltrimethoxysilane, silanetriol, and any combination thereof. In some variations, the zinc oxide is surface treated with ethoxylated alkyl silane. In a certain variation, the zinc oxide is surface treated with triethoxycaprylyisilane.

In certain embodiments, the compositions described herein include one type of surface-treated zinc oxide. In other embodiments, the compositions include a combination of two or more types of surface-treated zinc oxide. In one embodiment, a combination of surface-treated zinc oxide was used in the composition, and was chosen to balance efficacy, cost, skin feel and transparency on the skin. Examples of surface-treated zinc oxide suitable for use in the compositions described herein include two types of zinc oxide (and) triethoxycapryl ylsilane.

In some embodiments, the total concentration of zinc oxide (exclusive of any surface treatment agents or dispersion/slurries/pastes excipients) in the composition is less than or equal to 25%, less than 24%, less than 23% , less than 22%, less than 21%, less than 20%, less than 15%, or less than 10% by weight based on a total weight of the composition. In some embodiments, the concentration of a first zinc oxide in the composition is about 25%, about 24%, about 23%, about 22%, about 21%, about 20%, about 19%, about 18%, about 17%, about 16%, about 15%, about 14%, about 13%, about 12%, about 11 or about 10%, by weight, while the concentration of a second zinc oxide in the composition is about 15%, about 14%, about 13%, about 12%, about 11%, about 10%, about 9%, about 8%, about 7%, about 6%, about 5%, about 4%, about 3%, about 2%, about 1% or about 0%.

For example, in one variation, the composition includes a first zinc oxide treated with triethoxycaprytylsilane, Such first zinc oxide treated with triethoxycaprytylsilane may have a first weight ratio of zinc oxide to triethoxycaprytylsila.ne of 96.20:3.80. In other variations, the first zinc oxide treated with triethoxycaprylyisitane may have a median particle size (D50) of about 1790 nm. In yet other variations, the first zinc oxide exclusive triethoxycaprylylsilane is present in an amount of about 25 wt % based on the total weight of the composition.

In other variations, the composition further includes a second zinc oxide treated with triethoxycaprylylsilane. Such second zinc oxide treated with triethoxycaprylylsilane may have a second weight ratio of zinc oxide to triethoxycaprylylsilane being 96.67:3.33. In other variations, the second zinc oxide treated with triethoxycaprylylsilane has a median particle size (D50) of about 2880 nm.

In yet other variations of the foregoing compositions, a first zinc oxide treated with triethoxycaprylylsilane is present in an amount of about 18 wt % and a second zinc oxide treated with triethoxycaprylylsilane is present in an amount of about 7 wt % based on the total weight of the composition.

The foregoing compositions rm.ty further include one or more film formers and one or more SPF boosters, which are known to those skilled in the art.

In certain embodiments, provided is a hybrid water-in-silicone/oil inverted emulsion comprising: antioxidant components as disclosed herein, no more than 25 wt % zinc oxide based on a total weight of the emulsion, wherein the zinc oxide is surface treated; one or more film formers; and one or more SPF boosters.

In certain embodiments of the foregoing, the compositions described have a water resistance for up to about 80 minutes, or about 40 minutes.

In other embodiments that may be combined with any of the foregoing, the compositions described have a UVA Protection Factor (UVAPF) of at least ⅓ of the SPF value. In some embodiments, the compositions described have a UVAPE of at least 16.67, at least 17.16, or at least 20 at a critical wavelength of over 370 nm.

In other variations of the foregoing, the compositions described are formulated as a lotion or a cream. Such lotion/cream is suitable for use on human skin.

Dermatologically Acceptable Owner

In some variations, the compositions described herein further include a dermatologically acceptable carrier for active ingredients. The phrase “dermatologically acceptable carrier”, as used herein, means that the carrier is suitable for topical application to the skin, has good aesthetic properties, is compatible with the actives of the present invention and any other components, and will not cause any safety or toxicity concerns. A safe and effective amount of carrier is from about 50% to about 99.99%, preferably from about 60 to about 99.9%, more preferably from about 70% to about 98%, and even more preferably from about 80% to about 95% of the composition.

The carrier can be in a wide variety of forms. For example, emulsion carriers, including, but not limited to, oil-in-water, water-in-oil, silicone-in-water, water-in-silicone, water-in-oil-in-water, and oil-in-water-in-silicone emulsions, are useful herein.

Preferred carriers comprise an emulsion such as oil-in-water emulsions and water-in-oil emulsions, e.g., silicone-in-water or water-in silicone emulsions. As will be understood by the skilled artisan, a given component will distribute primarily into either the water or oil phase, depending on the water solubility/dispensability of the component in the composition. Oil-in-water emulsions are especially preferred.

Film Formers

In some variations, the compositions described herein further include one or more film formers, The film formers may include bis-octyldodecyl dimer dilinoleate/propanediol copolymer, octyldodecyl/glyceryl hydroxy stearate dilinoleate dimethicone copolymer, and any combination thereof.

In some embodiments, the one or more film formers are present in an amount of about 10%, about 7.5%, about 5%, about 4%, about 3%, about 2%, about 1%, about 0.5%, about 0.25%, or about 0.1% by weight. In certain embodiments, the one or more film formers are present in an amount of about 3 wt % based on the total weight.

SPF Boosters

In some variations, one or more SPF boosters are present in the compositions described herein. In some variations, a combination of SPF boosters is used.

Suitable SPF boosters may include butyloctyl salicylate, ethylhexyl methoxycrylene, styrene/acrylates copolymer (and) acrylates copolymer, glycogen, acrylates/methacryloyloxyethyl phosphate copolymer, dimethyl capramide, neopentyl glycol diethylhexanoate (and) neopentyl glycol diisostearate, daucus carota sative (carrot) root extract (and) helianthus annuus (sunflower) seed oil, argania spinose kernel oil (and) tocopheryl acetate (and) bisabolol, PVP (and) VP/Eicosene copolymer, VP/eicosene copolymer, hydrophobically modified starches, silica beads, PMMA beads, borosilicate beads, polyurethane beads, diatomaceous earth, betonite and hectorite clays and any combination thereof.

In some embodiments, the one or more SPF boosters are present in an amount of about 10%, about 9%, about 8%, about 7%, about 6%, about 5%, about 4%, about 3%, about 2%, about 1%, about 0.5%, about 0.2%, or about 0.1% by weight based on the total weight of the composition. In certain embodiments, the one or more SPF boosters are present in an amount of about 6 %. In certain embodiments, the one or more SPF boosters are present in an amount ranging from 0.1 wt % to 8 wt %.

### Methods of Use

Antioxidants function to neutralize reactive oxygen species (ROS) by quenching the excess energy on excited molecules or by scavenging free radicals generated by solar radiation. If the right type and level are present within skin where ROS are being formed, it is believed that antioxidants would be able to neutralize ROS before they can attack and oxidize other biomolecules or neutralize after its formation.

Antioxidant compositions detailed herein, including any personal care composition provided herein comprising an antioxidant described herein and/or combinations thereof, may be used in methods of administration and treatment as provided herein. In some embodiments, the present disclosure provides methods of preventing and/or treating photodamage induced by long UVA and/or visible light, comprising applying the compositions as described herein to a subject in need thereof.

In some embodiments, provided herein is a method of reducing skin darkening from long UVA and/or visible light, comprising topically applying the compositions as described herein. In some embodiments, provided herein is a method of reducing or treating melanogenesis from long UVA and/or visible light, comprising topically applying the compositions as described herein. In some embodiments, provided herein is a method of reducing free radical production from long UVA and/or visible light, comprising topically applying the compositions as described herein. In some embodiments, provided herein is a method of reducing photodamage from long UVA and/or visible light, comprising topically applying the compositions as described herein. In some embodiments, provided herein is a method of reducing ROS from long UVA and/or visible light, comprising topically applying the compositions as described herein.

Also provided is a method of suppressing immediate skin darkening (immediate after exposure) from long UVA and/or visible light, comprising topically applying the compositions as described herein. In certain embodiments, the composition suppresses delay tanning in dark skin, such as Fitzpatrick Skin phototypes (SPT) V and VI.

Also provided is a method of suppressing delay tanning (e.g., 7 days after exposure) from long UVA and/or visible light, comprising topically applying the compositions as described herein. In certain embodiments, the composition suppresses delay tanning in dark skin, such as Fitzpatrick Skin phototypes (SPY) V and VI.

Also provided is a method of decreasing erythema irnrnediately fromfroiri long UVA and/or visible light, comprising topically applying the compositions as described herein. In certain embodiments, the composition decreases erythema immediately in Caucasian skin, such as Fitzpatrick Skin phototypes (SPT) I, II and III.

Further provided, in certain embodiments, is a method of reducing increase in inflammation (e.g., reduction of COX-2 mediators) and/or induction of melanin (e.g., reduction of Mart-1 mediators) by long UVA and/or visible light, comprising topically applying the compositions as described herein.

In other aspects, the present invention provides a method for confirming antioxidant activity of a composition formulated for topical application to skin, wherein the method comprises clinically testing the composition for ability to reduce photodamage induced by long UVA and/or visible light. The invention also provides a method for screening compositions to be topically applied to skin for antioxidant behavior, wherein the screening method comprises determining the composition's ability to reduce photodamage induced by long UVA and/or visible light.

### EXAMPLES

The following Examples are merely illustrative and are not meant to limit any aspects of the present disclosure in any way.

### Example 1

### Evaluationof Long UVA and Visible Light Induced Biological Effects

This study was designed to determine the time course and measure the biolofical effects of long UVA radiation and visible light administered on the lower backs of male and female volunteers with melanocompetent and non-melanocompetent skin. The data below further demonstrates the protective effect of an exemplary antioxidant composition.

MATERIALS AND INSTRUMENTS. Three exemplary antioxidant compositions were tested, which are composed of Oxynex® ST Liquid (INCI: Diethylhexyl Syringylidene Malonate (and) Caprylic/Capric Triglyeride) at 0.5%, 1% and 2% respectively, in addition to 0.25% of Vitamin E (Tocopherol) and 0.01% of Vitamin C (Ascorbyl Palmitate NF). These compositions were referred as super antioxidant combinations (SOX) B1, B2 and B3.

In order to study the effectiveness of the antioxidant compositions, a specific solar simulator was developed. It was composed of a Xenon Lamp and a combination of optical filters to provide a solar simulator composed of ˜2% of long UVA (>370 nm up to 400 nm) and 98% of Visible Light (400 nm to 700 nm). A diffuse reflectance spectrometer was used to assess skin chromophores before and after skin exposure to a fixed amount of light (either 320 J/cm² or 480 J/cm²). The study showed that there is synergism between small amounts of UVA1 and Visible light further enhancing the skin pigmentation.

Skin erythema and melanin intensities were evaluated using diffuse reflectance spectroscopy in the visible range. The instrumentation employed for measurement of diffuse reflectance has been described and known in the art (Knoefel W T, Kollias N, Rattner D W, Nishioka N S, Warshaw A L: Reflectance spectroscopy of pancreatic microcirculation. J Appl Physiol 80:116-123, 1996). Briefly, a spectrophotometer and EQ-99X LDLS light source were used. Light was delivered and collected using a fiber optic probe that had multiple, tightly packed together, randomly arranged optical fibers with 50 μm core diameter providing a total delivery/collection spot diameter of 2.5 mm. Diffuse reflectance spectra were measured in the range 360 to 820 nm. Prior to data acquisition, a reference spectrum was measured on a BaSO₄ diffuse reflectance standard and all skin spectra were subsequently divided by this reference spectrum to ensure proper data calibration. Skin color measurements were made with a chromameter device and skin color parameters were recorded in the Commission International d'Eclairage L*a*b* colorimetric system (Billmeyer FW, Saltzman M: Principles of Color Technology, 2nd edn. New York: Wiley-Interscience, 1981; and Weatherall I L, Coombs BD: Skin color measurements in terms of CIELAB color space values. J Invest Dermatol 99:468-473, 1992).

Oxy-Hb and deoxy-Hb concentrations were also calculated from the absorbance spectra after correction for melanin and scattering contributions according to a previously described algorithm in the art (Kollias N, Baqer A: On the assessment of melanin in human skin in vivo. Photochem Photobiol 43:49-54, 1986; Stamatas G N, Kollias N. Blood stasis contributions to the perception of skin pigmentation. J. Biomed Opt 9:315-22 (2004); and Wagner J K, Jovel C, Norton H L, Parra E J, Shriver M D. Comparing quantitative measures of erythema, pigmentation and skin response using reflectometry. Pigment Cell Res 15:379-84 (2002)). Briefly, the apparent concentration of melanin was calculated from the slope of the absorption curve in the region 630-700 nm. Then the absorption spectra were corrected for the melanin contribution and were fitted for oxy-Hb and deoxy-Hb in the region 560-580 nm, where hemoglobins exhibit maxima. The contribution of deoxy-Hb absorption in the region 630-700 nm was taken into account and was subtracted from the initial estimation of melanin concentration. The process of correcting the absorbance curve for melanin and fitting for oxy-Hb and deoxy-Hb in the 560-580 nm region was repeated a second time. These concentration values of oxy-Hb and deoxy-Hb were finally recorded. The calculated concentrations were referred to as “apparent” because they are based on light absorption curves and were given in relative units. They are expected to be linearly related to the “absolute” concentrations of hemoglobins in units of mass per volume of tissue. Oxy-Hb values were used as a measure of erythema intensity and the melanin from the reflectance slope at the long wavelengths.

Readings of skin reflectance were taken in triplicate in the exposed and near area non-exposed and the difference of Oxy-hemoglobin (for erythema) and pigmentation were calculated for each time point and treatment.

STUDY DESIGN. The study was a double blind, placebo-controlled, single center study with a single application of multiple test products including active ingredients and placebo followed by single exposure of UVAI/Visible light at a dose of 320 J/cm². Up to 20 subjects were enrolled in the study and comprised of 10 subjects with Fitzpatrick Skin types (SPT) I-III and 10 subjects with SPT IV-VI (outlined in Table A). The total duration of the study was up to 7 days. The study consisted of 3 site visits. During the baseline visit (Day 0), subjects provided informed consent. Subject eligibility criteria were reviewed. Eligible subjects underwent baseline skin evaluation. Subjects was also exposed to the test product and placebo and irradiated with UVA/visible light.

TABLE A
Fitzpatrick Skin types (SPT)

| | |
|---|---|
| I | Always burns easily and severely (painful burn); tans little |
| | or none and peel |
| II | Usually burns easily and severely (painful burn); tans |
| | minimally or lightly, also peels |
| III | Burns moderately and tans about average |
| IV | Burns minimally, tans easily, and above average with each |
| | exposure |
| V | Rarely burns, tans easily and substantially |
| VI | Never burns and tans profusely |

At the baseline (Day 0) visit, each subject had 11 exposure sites to which either placebo, the active formula, or not treatment was applied. After 60 minutes of formula exposure, all 11 sites were exposed to an irradiation of UVA/visible light at a dose of 320 J/cm². Visit 1 (Day 0) also included clinical and noninvasive assessment of pigmentation and erythema. Subjects returned to the site for visit 2 (Day 1) for 3-punch biopsies of the exposed areas. Subjects underwent clinical and non-invasive observations. Biopsy samples were evaluated histologically for cutaneous effects in terms of DNA damage, free radical production and morphology. Subjects returned to the site on visit 3 (Day 7) for final clinical and non-invasive observations in addition to removal of sutures from biopsy. Skin evaluation was conducted by the trained evaluator for potential adverse events.

SUBJECT SELECTION. A total of 20 subjects were enrolled in this study. The subjects were comprised of 10 subjects with SPT IV-VI and 10 subjects with SPT I-III.

STUDY PROCEDURES AND EVALUATIONS. The study was a double blind, placebo-controlled, single center study with a single application of multiple test products including active ingredients and placebo followed by single exposure of UVA/Visible light 60 minutes post application. The total study duration per subject was 7 days,

There were a total of 3 site visits. The first visit was at baseline (Day 0) to assess whether subject qualifies to participate in the study. Baseline visit will also include exposure of the active formula, placebo, or no treatment and irradiation with UVA/Visible light. On visit 2 (Day 1) punch biopsies were performed on 3 exposure sites. All 3 visits (Day 0, Day 1, and Day 7) involved clinical and noninvasive skin evaluation by a trained evaluator who was overseen by licensed dermatologist. The final visit (Day 7) consisted of removal of stitches from biopsy performed on visit 2. Any adverse events reported were recorded by the study staff during the course of the study. This study was overseen by a dermatologist investigator.

Recruiting and Pre-Screening

Subjects were recruited using an IRB-approved material. Interested candidates were report to the test facility.

Baseline Visit 1 (Day 0)

The subject came to the research site without having applied any product to the face and body in the morning (except the morning wash). The eligibility criteria (Inclusion/Exclusion) and Dermatological Medical History were completed for each potential subject. Subjects who met all eligibility criteria was assigned a subject number in sequential order depending on Fitzpatrick skin type.

Clinical examination of baseline cutaneous state of the back was performed by a trained evaluator who was overseen by the dermatologist investigator.

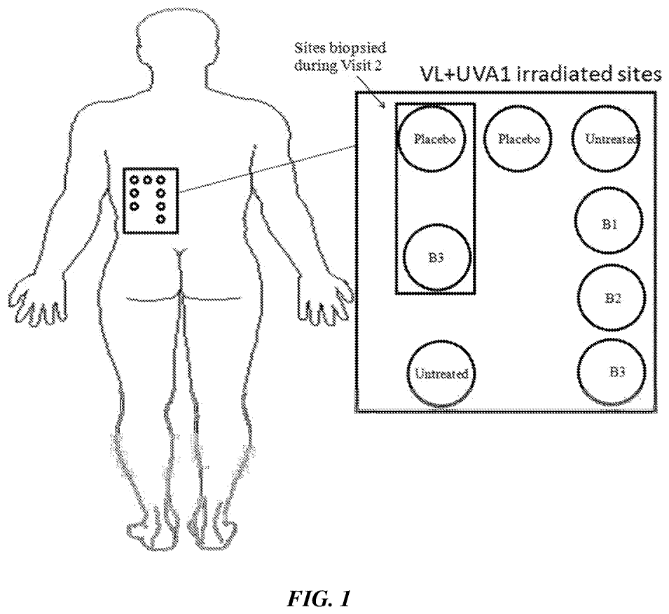

The subject underwent exposure of the test product, placebo, and no treatment on the lower back by full occlusion for 60 minutes using a 12-mm Finn Chamber before irradiation. The topical compositions used are outlined in Table 1. Each subject had a total of 8 exposure sites as outlined in FIG. 1.

TABLE 1
Ingredients of Topical Compositions

| Compositions- | | |
|---|---|---|
| each in a 70/30 (Ethanol:Propylene | | |
| Glycol) vehicle | UV Filters | # of sites |
| Placebo | No | 2 |
| Untreated | None | 1 |
| SOX B1 (0.5% Oxynex ® ST Liquid | No | 1 |
| SOX B2 (1% Oxynex ® ST Liquid) | No | 1 |
| SOX B3 (2% Oxynex ® ST Liquid) | No | 2 |

Upon 60 minutes of exposure to the or placebo, the Finn Chamber was removed from the subject's back. Subsequently, a sheet of aluminum with adhesive backing and 12-mm punch holes was applied onto the exposed area prior to irradiation. The LS 1000 Solarlight® Simulator was modified to emit 370 nm-700 nm UVAI/Visible light at a fluence rate of approximately 150 mW/cm² for a total dose of 320 J/cm². Upon completion of irradiation, the exposed areas were immediately assessed (clinically/non-invasively) for pigmentation and erythema. In addition, subjects underwent visual examination (Table 2), global assessment by the investigator, diffuse reflectance spectroscopy, and photography of the exposed areas.

TABLE 2
Visual Exam: Investigator's Global Assessment

| IGA | Description of Erythema |
|---|---|
| 0 | Clear of erythema |
| 1 | Trace erythema |
| 2 | Visible, not confluent erythema, no sharp borders |
| 3 | Confluent erythema with 4 sharp borders |
| 4 | Intense erythema |
| IGA | Description of Hyperpigmentation |
| 0 | Clear of hyperpigmentation |
| 1 | Almost clear of hyperpigmentation |
| 2 | Mild, but noticeable hyperpigmentation |
| 3 | Moderate hyperpigmentation (medium brown in quality) |
| 4 | Severe hyperpigmentation (dark brown in quality) |
| 5 | Very severe hyperpigmentation (very dark brown, almost |
| | black in quality) |

The above Investigator's Global Assessment (IGA) scale was used it immediately following irradiation and study days 2 and 3 for Immediate Pigment Darkening (IPD), persistent pigment darkening (PPD), and delayed tanning (DT).

Immediate Pigment Darkening occurred immediate following irradiation and fades within 20 minutes to 2 hours, Persistent Pigment Darkening became visible several hours following irradiation and remained visible between 2 and 24 hours after irradiation. Delayed tanning occurred days after irradiation.

Visit 2 (Day 1)

The exposed areas were assessed clinically and non-invasively for pigmentation and erythema. In addition, subjects underwent visual examination, global assessment by the investigator; diffuse reflectance spectroscopy and/or colorimetry, and photography of the exposed areas.

Following evaluation, three 4-mm skin punch biopsies from the exposed sites (refer to FIG. 1) were extracted per subject. These biopsies then underwent staining and histologic evaluation. The histologic parameters to be investigated included: hematoxylin & eosin, Fontana Masson, Melan A Cyclooxygenase (COX-2), 8-oxoguanine, cyclin D-1, and MMP-1. Table 4 outlines the histologic evaluation and the visible light effect it correlates to.

TABLE 3
Biopsy Markers

| Technique | UVAI/Visible Light Effects |
|---|---|
| Hematoxylin & Eosin (H&E) | Morphology and Melanin (quantitative |
| Fontana Masson stain (melanin) | analysis) |
| Melan A | Cell (DNA), damage, inflammation |
| Cyclooxygenase (COX-2) | Oxidative Stress |
| 8-oxoguanine | Proliferation |
| Cyclin D1 | Collagen Degradation |
| MMP1 | |

Visit 3 (on Day 7)

Subjects underwent suture removal from biopsies performed during visit 2. Exposed areas were assessed clinically and non-invasively for pigmentation and erythema. In addition, final visual examination, investigator's global assessment, and diffuse reflectance spectroscopy and/or colorimetry and photography of the exposed areas were performed.

Subject Instructions

Throughout the study, subjects agreed to comply with dates and hours of evaluation visits and practice sun protection measures; limit excessive UV exposure (including artificial UV), wear long sleeves, hats, and sunglasses for additional sun protection

Throughout the study, subjects agreed not (1) apply any product on the days of the visits to the research site; (2) apply any sunscreen or sunscreen-containing products during the study; and (3) modify the usual hygiene or care products and/or use new products throughout the study, including but not limited to, facial products, lip products, body products, hair products, perfumes, or laundry products.

Subjects were allowed during the study (except the day of the evaluation visits) to use their usual skin care products (face, body . . . ) and to use their usual cleansing products.

TEST ARTICLE ADMINISTRATION. Table B below lists description of test articles.

TABLE B
Test Article Description

| | |
|---|---|
| Test Product | SOX (Oxynex + 0.25% Vitamin E + 0.01% Vitamin C) |
| | Placebo: 0% |
| | All products in a 70:30 (ethanol:Propylene Glycol) |
| | vehicle |
| Storage | Before the beginning of the study, the test products |
| | are kept at a room temperature in a dedicated room. |
| | This room is locked and access controlled. The study |
| | treatment will be stored at the investigational site in |
| | accordance with Good Clinical Practice (GCP) and Good |
| | Manufacturing Practice (GMP) requirements and the |
| | instructions given by the Clinical Research Organization |
| | (CRO) on behalf of the sponsor, and will be inaccessible |
| | to unauthorized personnel. |
| Patch | Pipette 0.15 mL of product or placebo onto Finn |
| preparation | Chamber |
| Application | Lower back |
| site | |
| Application | Apply patch to lower back site of exposure |
| Instructions | |

DATA ANALYSIS AND RESULTS. An overall review of the data was performed by the investigators to identify any potential concerns associated with use of the test product under conditions of the study.

Clinical observation, non-invasive data (e.g., Oxy-Hemoglobin and Melanin) and biological markers have shown the following results in dark and light skin patients. In dark skin patients (SPT V and VI) statistically significant reduction of immediate skin darkening and/or skin pigmentation was observed when compared with placebo treated sites. This effect was observed at SOX B2 and B3. In dark skin patients (SPT V and VI) statistically significant induction of delayed skin pigmentation (7 days after the exposure) was observed when compared with placebo treated sites. This effect was observed at SOX B2 and B3. In light skin patients (SPT I to III) statistically significant suppression of skin erythema (immediate after the exposure) was also observed at SOX B2 and B3.

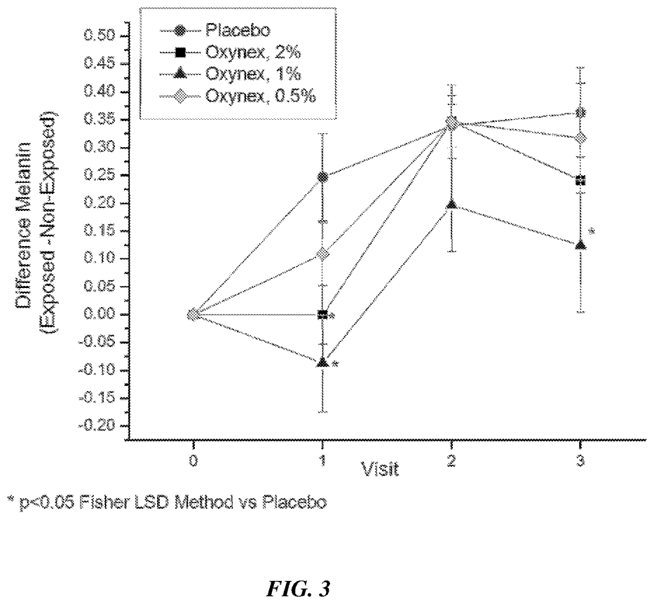

FIG. 3 illustrates the difference in the mean melanin content in skin (exposed—non exposed) for the 10 dark skin Phototypes (IV-VI) for each time point: 0 being the baseline, 1 as immediate after the exposure, 2 as 24 hours after the exposure and 3 as 7 days after the exposure. Graph illustrates the mean values and the standard deviation for each mean. All 10 subjects with skin phototype IV-VI had an immediate pigment darkening response after exposure to long UVA1 and Visible Light. The difference in skin pigmentation (as measure by Melanin or delta L in Chromameter, graph not shown here) demonstrated that the site that was treated with the two highest concentration of SOXs was significantly lighter immediately after irradiation, time 1 in the graph with <p=0.05. At day 7, the trend continued and we observe significant reduction of delay tanning in skin on the sites treated with two highest concentration of SOXs when compared with placebo. This analysis was performed using a General Linear Statistical Model in Minitab by Fisher's least significant difference.

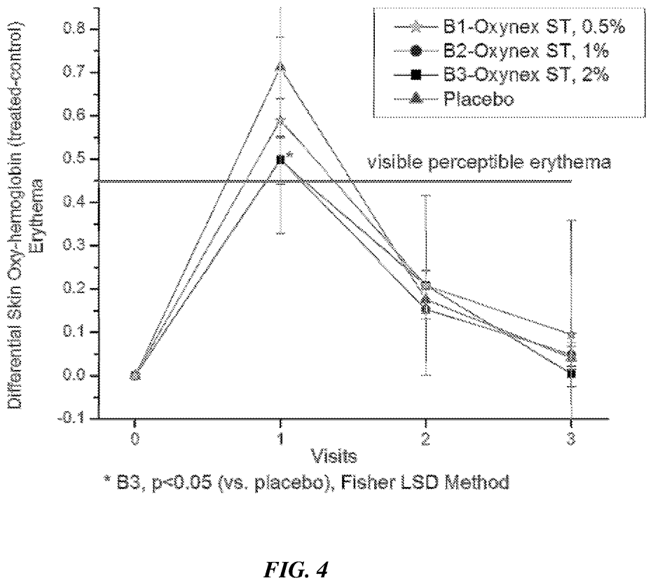

FIG. 4 illustrates the difference in the mean erythema content in skin (exposed—non exposed) for the 10 light skin Phototypes (I-III) for each time point: 0 being the baseline, 1 as immediate after the exposure, 2 as 24 hours after the exposure and 3 as 7 days after the exposure. Graph illustrates the mean values and the standard deviation for each mean. All 10 subjects with skin phototypes I-III had an immediate erythema after exposure to long UVA1 and Visible light. The difference in skin erythema (as measure by oxyhemoglobin or delta a in Chromameter, graph not shown here) demonstrated that the site that was treated with the highest concentration of SOXs was significantly lighter immediately after irradiation, time 1 in the graph with <p=0.05. No significant erythema is observed 24 hours after exposure in any treatment sites. This analysis was performed using a General Linear Statistical Model in Minitab by Fisher's least significant difference.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1.-55. (canceled)

56. A sunscreen composition, wherein the composition comprises;
one or more mineral sunscreen actives; and
diethylhexyl syringylidene malonate,
and wherein the composition is effective to protect skin of a subject front long Ultraviolet A and/or visible light.

57. The composition of claim 56, wherein the composition further comprises an organic sunscreen active ingredient.

58. The composition of claim 56, wherein the composition does not comprise any organic sunscreen active ingredients.

59. The composition of claim 56, wherein the composition further comprises one or both of vitamin C and vitamin E.

60. The composition of claim 56, wherein the amount of diethylhexyl syringylidene malonate is more than 0.5 wt % of a total weight of the composition.

61. The composition of claim 56, wherein the one or more mineral sunscreen actives comprise zinc oxide.

62. The composition of claim 61, wherein an amount of zinc oxide is not higher than 25 wt % of a total weight of the composition.

63. The composition of claim 61, wherein the zinc oxide comprises zinc oxide surface-treated with triethoxycaprylylsilane.

64. The composition of claim 63, the zinc oxide comprises a first zinc oxide surface-treated with triethoxycaprylylsilane which has a first weight ratio of zinc oxide to triethoxycaprylylsilane of 96.20:3.80.

65. The composition of claim 64, wherein the first zinc oxide surface-treated with triethoxycaprylylsilane has a first median particle size (D50) of about 1790 nm.

66. The composition of claim 64, wherein the surface-treated zinc oxide further comprises a second zinc oxide surface-treated with triethoxycaprylylsilane which has a second weight ratio of zinc oxide to triethoxycaprylylsilane of 96.67:3.33.

67. The composition of claim 66, wherein the second zinc oxide surface-treated with triethoxycaprylylsilane has a second median particle size (D50) of about 2880 nm.

68. The composition of claim 56, wherein the composition further comprises one or more film formers comprising one or both of bis-octyldodecyl dimer dilinoleate/propanediol copolymer and octyldodecyl/glyceryl hydroxy stearate dilinoleate dimethieone copolymer.

69. The composition of claim 56, wherein the composition further comprises one or more SPF boosters which comprise one or both of butyloctyl salicylate and ethylbexyl methoxycrylene.

70. The composition of claim 56, wherein the composition further comprises one or more silicone-based emulsifiers comprising cetyl PEG/PPG 10/1 Dimethieone.

71. A super antioxidant composition for topical application, wherein the composition comprises or consists essentially of
diethylhexyl syringylidenc malonate;
vitamin C; and
vitamin E.
and wherein the super antioxidant composition is effective to protect skin of an subject from long UVA and/or visible light.

72. A method of reducing skin darkening caused by UVA and/or visible light or a method of reducing or treating melanogenesis caused by UVA and/or visible light, wherein the method comprises topically applying to skin the composition of claim 56.

73. A method of reducing photodamage caused by long UVA and/or visible light or a method of reducing reactive oxygen species (ROS) caused by long UVA and-or visible light, wherein the method comprises topically applying to skin the composition of claim 56.

74. A method of suppressing delayed tanning and erythema in human skin caused by long UVA and/or visible light, wherein the method comprises topically applying to skin the composition of claim 56.

75. A method of decreasing erythema caused by long UVA and/or visible light immediately in human skin, wherein the method comprises topically applying to skin the composition of claim 56.
